(19) United States
(12) Patent Application Publication
(10) Pub. No.: US 2023/0324263 A1
(43) Pub. Date: Oct. 12, 2023

(54) ANALYZER WITH A POSITIVE DISPLACEMENT PUMP AND A VALVE AND ANALYSIS PROCESS WITH SUCH AN ANALYZER

(71) Applicant: Dräger Safety AG & Co. KGaA, Lübeck (DE)

(72) Inventors: Jens REKOW, Lübeck (DE); Marie-Isabell MATTERN-FRUEHWALD, Lübeck (DE); Tobias REIER, Lübeck (DE); Luis Brunswick FRANCO, Lübeck (DE); Nick BENDORF, Lübeck (DE)

(21) Appl. No.: 18/194,683

(22) Filed: Apr. 3, 2023

(30) Foreign Application Priority Data
Apr. 7, 2022 (DE) 10 2022 108 432.9

(51) Int. Cl. G01N 1/24; G01N 33/98

(52) U.S. Cl. CPC G01N 1/24; G01N 33/98; G01N 2001/2244

(57) ABSTRACT

An analyzer and a process analyze a breath sample exhaled by a subject for a predetermined substance, particularly alcohol. An input fluid connection connects an input unit (1) to a measuring chamber (3). A suction fluid connection connects the measuring chamber to a suction chamber unit (5, 6), that is selectively transferrable to a with minimum volume state or a maximum volume state. A sensor (12) measures an amount or a concentration of the substance in the measuring chamber. A drive unit (4, 11) moves a valve (2, 13) for the input fluid connection selectively into a closing or into a releasing end position. The drive unit can also move the suction chamber unit between the two states. The movement of the valve (2, 13) from one end position to the other end position is coupled with a transfer of the suction chamber unit (5, 6) between the two states.

### CROSS REFERENCE TO RELATED APPLICATIONS

This application claims the benefit of priority under 35 U.S.C. § 119 of German Application 10 2022 108 432.9, filed Apr. 7, 2022, the entire contents of which are incorporated herein by reference.

### TECHNICAL FIELD

The invention relates to an analyzer (an analysis device) and a process for analyzing a gas sample provided (input) by a subject, in particular an exhaled breath sample, for a predetermined substance, in particular for alcohol. Such an analyzer can be used for checking whether or not the subject has ingested alcohol, in particular ethyl alcohol (ethanol) and optionally admixtures of other alcohols. If the subject has consumed alcohol above a detection limit, his or her blood contains alcohol and therefore the air exhaled by the subject contains breath alcohol. Optionally, such an analyzer can be used to determine what the breath alcohol concentration is in his or her breath, and from this the amount of alcohol in his blood can be deduced.

### BACKGROUND

In the following, the term “alcohol” is used for a substance to be detected in the blood of the subject, and the term “breath alcohol” is used for a substance which is then contained in a gas sample, in particular breath sample, of the subject if his or her blood contains alcohol.

Various portable breath alcohol analyzers have become known, for example from DE 10 2017 008 008 A1. A subject introduces a breath sample into a mouthpiece of the analyzer. At least a portion of the breath sample is directed to a measuring chamber in the analyzer. An electrochemical sensor measures at what concentration or in what amount the gas coming from the breath sample and being in the measuring chamber contains breath alcohol. Of course, the measurement can lead to the result that no breath alcohol above a detection limit is contained in the gas and therefore in the breath sample.

### SUMMARY

The invention is directed to the object of providing an analyzer and a process for analyzing a gas sample for a predetermined substance, wherein the gas sample is from a subject to be analyzed for a predetermined substance, and wherein the analyzer and process are intended to be more reliable than known analyzers and processes.

The object is accomplished by an analyzer having analyzer features according to the invention and by a process having process features according to the invention. Advantageous embodiments of the analyzer according to the invention are, as far as useful or reasonable, also advantageous embodiments of the process according to the invention and vice versa.

The analyzer according to the invention and the process according to the invention are capable of analyzing a gas sample for a given substance or a set of given substances. A subject, in particular a human, has delivered this gas sample, in particular exhaled. The substance is, in particular, breath alcohol or another drug or other addictive substance which can be detected in a gas sample from a subject.

The analyzer comprises an input unit. The subject can input the gas sample into this input unit, in particular exhale a breath sample, or the gas sample can be taken up by the input unit in another way. The input unit is permanently or at least temporarily connected to the rest of the analyzer, preferably detachably (releasably) connected.

Further, the analyzer includes a measuring chamber and provides an input fluid connection. A “measuring chamber” is to be understood to mean a cavity of the analyzer, wherein the cavity can receive a gas sample. The input fluid connection at least temporarily connects the input unit to the measuring chamber so that, when the fluid connection is established, a fluid, in particular a quantity of the gas sample input into the input unit, can flow from the input unit into the measuring chamber and preferably but not necessarily vice versa from the measuring chamber back into the input unit.

A sensor of the analyzer is capable of measuring an indicator of the amount and/or an indicator of the concentration of the predetermined substance in a gas. The sensor is at least able to determine whether the amount or concentration is below or above a predetermined threshold, e.g. a detection limit. The gas that the sensor examines for the substance is located in or at the measuring chamber. The sensor is preferably arranged in the measuring chamber or on or in at least one wall of the measuring chamber. The sensor is capable of outputting a signal that correlates with the amount and/or concentration of the substance.

Furthermore, the analyzer comprises a suction chamber unit, for example with a bellows and a plate, the plate being capable of expanding and compressing the bellows and thereby changing the volume of the space enclosed by the bellows, or with a piston-cylinder unit. A suction fluid connection at least temporarily connects the suction chamber unit to the measuring chamber. A fluid can flow from the measuring chamber through this suction fluid connection into the suction chamber unit and back from the suction chamber unit through this suction fluid connection into the measuring chamber.

Notes:
A “fluid connection” (“fluid communication”) is established between a first component and a second component if a fluid, in particular a gas, can flow from the first component to the second component. It is possible that the first component is directly connected to the second component and the fluid connection is established with the aid of two overlapping openings in the two components.
It is also possible that the fluid connection is established with the aid of a fluid guide unit, whereby the fluid guide unit connects the two components to one another, and a fluid can flow through the fluid guide unit. A “fluid guide unit” is understood to be a component that is capable of guiding a fluid along a trajectory determined by the geometry and position of the component, and ideally preventing the fluid from leaving this trajectory. A tube and a hose are two examples of a fluid guiding unit.
The analyzer according to the invention establishes the input fluid connection and the suction fluid connection each at least temporarily during a use of the analyser. It is possible that neither of the two fluid connections is established in an idle state of the analyzer.

The suction chamber unit fluid-tightly encloses (encircles) a space, except for a fluid connection described below, and can be selectively transferred into a minimum volume state or into a maximum volume state. During transfer into the minimum volume state, a fluid is conveyed out of the suction chamber unit and forced into the suction fluid connection. This causes fluid to be forced out of the suction fluid connection and into the measuring chamber. This event forces (expels) fluid out of the measuring chamber and into the input fluid connection or into a separate output fluid connection. The process of forcing fluid out of the measuring chamber therefore causes the measuring chamber to be purged (cleaned). In particular an old gas sample is removed out of the measuring chamber.

During the transfer into the maximum volume state, a fluid is sucked into the suction chamber unit. This causes fluid to be sucked through the suction fluid connection into a chamber of the suction chamber unit. As a result, fluid is drawn from the environment and/or input unit through the input fluid connection into the measuring chamber. Preferably, at least a portion of this fluid originates from the input unit and contains gas delivered by the subject. Thus, the process of transferring the suction chamber unit into the maximum volume state causes at least a portion of the gas sample delivered by the subject to be drawn or sucked into the measuring chamber.

The analyzer further comprises a valve. This valve can be moved either into a closing end position or into a releasing end position. In the closing end position, the valve closes the input fluid connection and thereby interrupts the input fluid connection. Of course, even when the input fluid connection is closed, unavoidable gaps or slits can occur through which gas can pass from the input unit into the measuring chamber. In the releasing end position and optionally also in each intermediate position, the valve at least partially releases the input fluid connection.

Furthermore, the analyzer comprises a drive unit. Preferably, the drive unit comprises an actuator and a rod.

On the one hand, the drive unit can selectively move the valve into the closing end position or into the releasing end position. On the other hand, the same drive unit can selectively transfer the suction chamber unit into the maximum volume state or into the minimum volume state.

The drive unit is mechanically coupled to both a valve body (closure part) of the valve and a part of the suction chamber unit. One of the following effects is achieved by these two couplings:
A movement of the valve into the releasing end position is synchronized with a transfer of the suction chamber unit into the minimum volume state. Conversely, a movement of the valve into the closing end position is synchronized with a transfer of the suction chamber unit into the maximum volume state.
A movement of the valve into the closing end position is synchronized with a transfer of the suction chamber unit into the minimum volume state. Conversely, a movement of the valve into the releasing end position is synchronized with a transfer of the suction chamber unit into the maximum volume state.

The process according to the invention is carried out using an analyzer according to the invention and comprises the following steps:
Initially, the valve is in the closing end position and interrupts the input fluid connection.
A gas sample enters into the input unit or is taken up by the input unit.
The drive unit moves the valve into the releasing end position so that the valve releases the input fluid connection.
The drive unit transfers the suction chamber unit into the maximum volume state. This transfer sucks gas from the input unit through the input fluid connection into the measuring chamber.
The drive unit then transfers back the suction chamber unit to the minimum volume state. This back transfer causes gas to be conveyed from the suction chamber unit through the suction fluid connection into the measuring chamber. This in turn ejects gas from the measuring chamber, which flushes (purges) the measuring chamber.
The drive unit moves the valve back into the closing end position.

The order in which these steps are listed is not necessarily the chronological order in which these steps are performed. As far as reasonable, a different order is possible.

Furthermore, the process comprises the following step: The sensor measures an indicator of the concentration and/or amount of the substance, in particular of breath alcohol, in the gas that is in the measuring chamber. At least a portion of this gas in the measuring chamber, ideally all gas in the measuring chamber, belongs to the gas sample and originates from the input unit and therefore from the subject.

In a first alternative of the invention, the following two steps are performed simultaneously:
The valve is moved into the releasing end position.
The suction chamber unit is transferred into the minimum volume state.

According to the first alternative, the following two steps are also performed simultaneously:
The valve is moved into the closing end position.
The suction chamber unit is transferred into the maximum volume state.

In a second alternative of the invention, the following two steps are performed simultaneously:
The valve is moved into the closing end position.
The suction chamber unit is transferred into the minimum volume state.

According to the second alternative, the following two steps are also performed simultaneously:
The valve is moved into the releasing end position.
The suction chamber unit is transferred into the maximum volume state.

The characteristic that two operations are performed simultaneously includes the possibility that a difference within a tolerance band occurs between the start times or end times of each of the two operations.

By means of the analyzer and the process according to the invention, at least one gas sample is sucked from the input unit through the input fluid connection into the measuring chamber. This gas sample is a part of that quantity of gas which the subject has delivered into the input unit or has otherwise entered the input unit. As already explained, the gas sample is sucked into the measuring chamber by transferring the suction chamber unit into the maximum volume state. Preferably, the input unit is connected to the rest of the analyzer at least during the period in which the gas sample is sucked into the measuring chamber, so that it is ensured that the sucked-in gas sample originates from the gas that the subject has exhaled or otherwise input into the input unit.

Of course, the gas sample to be analyzed can only flow from the input unit into the measuring chamber for being analyzed there by the sensor if the valve is in the releasing end position or at least in an intermediate position. Furthermore, in many cases, the measuring chamber can only be flushed and made available for receiving another gas sample when the valve is in the releasing end position or at least in an intermediate position. Only then can gas flow out of the measuring chamber through the input fluid connection. It is also possible that gas from the measuring chamber does not pass through the input fluid connection but leaves the analyzer through a separate fluid connection.

With the valve in the closing end position, the measuring chamber is separated from the input unit and preferably from the environment, ideally in a fluid-tight manner. On the one hand, this reduces the risk of ambient conditions changing the sensor. In particular, the risk is reduced that particles or substances from the input unit or the environment affect the sensor or that deposits on the sensor occur. On the other hand, there is less risk that a component of the sensor will evaporate, for example an electrolyte. Both ambient conditions and evaporation can cause the sensor to deliver incorrect or unreliable measurement results or even fail.

For the reasons just stated, the valve must be temporarily in the releasing position, namely to allow a sample of the gas being tested to flow into the measuring chamber, and should otherwise be in the closing position.

According to the invention, the suction chamber unit sucks a sample of the gas sample to be examined into the measuring chamber by the suction chamber unit being transferred into the maximum volume state. Due to this feature, in many cases a defined amount of the gas sample to be analyzed is delivered into the measuring chamber in a relatively short time. In many cases it would be much more difficult to determine the amount of gas sample entering the measuring chamber if the amount depended significantly on the strength and duration with which the subject inputs the gas sample, on the volume flow rate at which the gas sample flows into the measuring chamber, or if the gas sample diffused into the measuring chamber. In addition, in many cases such a procedure to move a gas sample into the measuring chamber requires more time than the use of the suction chamber unit according to the invention.

In many cases, the measuring chamber contains almost only the gas that has been drawn into the measuring chamber by the suction chamber unit being transferred into the maximum volume state. The gas that enters the measuring chamber by diffusion or by the subject ejecting a gas sample while the suction chamber unit is at rest often is a negligible amount. The effect of the gas in the measuring chamber being almost entirely from suction facilitates ensuring in many cases that essentially only air from the subject's lungs enters the measuring chamber, but little air from the upper airways or mouth. This increases the reliability of the measurement, particularly when alcohol is to be detected in the subject's blood. In another application, this feature makes it easier to ensure that first air from the mouth and then air from the subject's lungs enters the measuring chamber and is analyzed there for the substance in each case, while largely preventing air from the upper airways from entering the measuring chamber.

Thanks to the invention, only one drive unit is required both to move the valve and to transfer the suction chamber unit. This saves one drive unit, and thus in many cases space and electrical energy, compared to an embodiment in which two different drive units are provided. In addition, only one drive unit needs to be supplied with electrical energy and controlled and monitored, rather than two.

According to the invention, the movement of the valve from one end position into the other end position is mechanically coupled to the transfer of the suction chamber unit from one state into the other state, in that the same drive unit is mechanically coupled to both the valve and the suction chamber unit. Thanks to this coupling, it is achieved in many cases that the valve
is only open as long as necessary, namely so that gas can flow into the measuring chamber, and
is closed for as long as possible, thereby separating the measuring chamber and the sensor from the environment and from the input unit.

Thanks to the mechanical coupling, there is no need to use an electronic or pneumatic control unit that couples the valve movement with the state transfer.

According to the invention, the input fluid connection connects the input unit to the measuring chamber. In one embodiment, the input unit comprises or defines a channel connecting the environment to the input fluid connection. Preferably, this channel tapers as seen in a direction towards the measuring chamber. In one implementation, the input unit further comprises a mouthpiece that can be detachably connected to a housing of the analyzer and guides a delivered gas sample, in particular a breath sample, towards the input fluid connection. These two implementations can be combined with each other.

According to the invention, the drive unit is able to both move the valve and transfer the suction chamber unit, thereby causing a synchronized movement of these two parts. Preferably, the drive unit comprises an actuator that is mechanically coupled to both the valve and the suction chamber unit.

According to the invention, the step of transferring the suction chamber unit into the maximum volume state effects the following: Gas from the input unit is sucked through the input fluid connections into the measuring chamber. Preferably, the measuring chamber is located between the input unit and the valve on one side and the suction chamber unit on the other side. Particularly preferably, the input unit, the input fluid connection, the measuring chamber, and the suction chamber unit are arranged one behind the other along a line. Preferably, the valve is also located on this line, namely between the input unit and the measuring chamber. The embodiment with the measuring chamber between the input unit and the suction chamber unit leads to a particularly space-saving and robust arrangement. In many cases, the preferred embodiment in which various components are arranged along a line accomplishes the following: The gas sample is conveyed linearly, which reduces the risk of unwanted turbulences.

In a preferred implementation, the drive unit additionally comprises a mechanical valve connection element. The valve connecting element preferably is or comprises a rod or bar. The valve comprises a closure part, which preferably functions as a valve body, and a closure part seat, for example a sealing ring. The closure part is movable relative to the closure part seat, preferably linearly movable in two opposite directions. When the valve is in the closing end position, the closure part is in contact with the closure part seat, preferably fluid-tight except for unavoidable slits and/or gaps. When the valve is in the releasing end position or in an intermediate position, a gap occurs between the closure part and the closure part seat. According to this implementation, the valve connecting element mechanically connects the actuator to the closure part. This implementation results in a particularly simple mechanical structure. The valve connecting element bridges the distance between the actuator and the closure part. This allows the actuator to be arranged at a distance from the closure part, which in some cases facilitates the positioning of the input fluid connection and the electrical supply of the actuator.

This embodiment can be combined with an embodiment in which the suction chamber unit is located between the measuring chamber and the actuator. Preferably, the measuring chamber is again located between the suction chamber unit and the input unit.

In another embodiment, the suction chamber unit comprises a variable volume suction chamber having a fluid-tight wall, such as a bellows. Further, the suction chamber unit comprises a mechanical chamber modifying element, such as a plate. It is also possible that the suction chamber unit comprises a piston-cylinder unit, wherein the piston is movable relative to the cylinder and the suction chamber is provided inside the cylinder and limited by the piston. The piston then acts as the chamber modifying element. A movement of the chamber modifying element relative to the suction chamber causes the volume of the suction chamber to change. The suction fluid connection according to the invention connects the suction chamber to the measuring chamber. The drive unit comprises an actuator and a mechanical chamber connecting element. This chamber connecting element connects the actuator to the chamber modifying element.

At least two of the just mentioned embodiments can be combined.

According to the invention, the drive unit is able to move the valve back and forth between the releasing and the closing end positions. In one embodiment, a volume flow sensor is capable of measuring an indicator of the volume flow of gas through the input fluid connection into the measuring chamber. A “volume flow” through a fluid connection is understood to be a volume per unit time of a fluid flowing through the fluid connection. For example, the volume flow sensor measures the difference in pressure at two different measurement positions, and an evaluation unit derives the volume flow from the pressure difference. According to this embodiment, the analyzer according to the invention is configured as follows: Depending on the measured volume flow, the analyzer is able to automatically trigger the step that the drive unit moves the valve into the closing end position (to actuate the drive unit to move the valve). This embodiment facilitates the introduction (guiding) of a defined and/or known amount of gas into the measuring chamber and the subsequent closing of the measuring chamber. This embodiment increases the reliability of the measurement result. The amount of gas can be derived from the measured volume flow.

According to the invention, the input fluid connection connects the input unit to the measuring chamber. In the releasing end position, the valve releases the input fluid connection. In one embodiment, a fluid guide unit, for example a tube, surrounds a closure part, e.g. a valve body, of the valve and optionally also a part of a valve connection element of the drive unit. A gap occurs between the fluid guide unit and the valve body, such as an annular gap. The input fluid connection passes through the fluid guide unit and includes this interstitial space. In one embodiment, a closure part seat of the valve is adjacent to this intermediate space.

This embodiment makes it possible in a particularly simple manner to design the valve so that a linear movement of the closure part (the valve body, e.g.) moves the valve from one end position to the other end position. The fluid guide unit protects to a certain extent the closure part from mechanical damage from the outside.

According to the invention, the step of transferring the suction chamber unit into the maximum volume state causes gas to be sucked through the input fluid connection into the measuring chamber. In one embodiment, the step of transferring the suction chamber unit into the minimum volume state causes gas to be conveyed, for example to be expelled, through the input fluid connection out of the measuring chamber. This step flushes the measuring chamber and enables it to receive a new gas sample. A separate fluid connection to purge the measuring chamber is possible, but not required.

In a preferred embodiment, the step of moving the valve into the releasing end position and thereby releasing the input fluid connection is triggered automatically and time-controlled or event-controlled. According to one implementation, the event is detected that inputting the gas sample into the input unit is started. For example, the event is detected that a mouthpiece or other input element has been placed on a base body of the analyzer or that gas is flowing into the input element. In a first realization form, the step of moving the valve into the releasing end position is started when a predetermined period of time has elapsed since the event that inputting or intaking of the gas sample has been started.

In a second realization form, the step of transferring the valve into the releasing end position is started when a predefined opening event has occurred since the start of inputting the gas. The opening event preferably depends on the volume or amount of the gas sample that has been input to the input unit so far and/or flows into the input unit.

Both realization forms of the preferred embodiment contribute to the fact that essentially only air from the subject's lungs flows into the measuring chamber, but not at all or only little air from the upper airway and the mouth. This effect increases the reliability of correctly determining the level of alcohol in the subject's blood by analyzing the gas sample for breath alcohol.

Gas can essentially only enter the measuring chamber when the valve is in the releasing end position. Usually, the valve is only in an intermediate position between the two end positions for a very short time. In one embodiment, it is ensured with higher reliability that the amount of gas in the measuring chamber is at least approximately known. In one embodiment, the amount of gas that has flowed into the measuring chamber so far is measured. When the measured quantity reaches a predetermined quantity threshold, the step of moving the valve back into the closing end position is triggered.

According to the invention, the drive unit transfers the suction chamber unit into the state maximum volume state and into the minimum volume state. Alternative configurations are possible as to the order in which these two steps are carried out.

In a first alternative, the suction chamber unit is in the maximum volume state before the procedure is carried out, i.e. before a gas sample to be analyzed enters the measuring chamber. First, the step of transferring the suction chamber unit to the minimum volume state is performed. This flushes the measuring chamber, in particular by removing gas from a previous gas sample from the measuring chamber. Then, the step of transferring back the suction chamber unit to the maximum volume state is performed. As a result, a quantity of the gas sample currently under investigation is sucked into the measuring chamber. The sensor now measures an indicator of the amount or concentration of the substance.

In a second alternative, the reverse sequence is performed. Before the procedure is carried out, the suction chamber unit is in the minimum volume state. First, the step of transferring the suction chamber unit into the maximum volume state is performed. As a result, a quantity of the gas sample currently being analyzed is drawn into the measuring chamber. The sensor measures an indicator of the amount or concentration of the substance. The suction chamber unit is then transferred back to the minimum volume state. This flushes the measuring chamber.

In a preferred embodiment, the analyzer comprises an input fluid guide unit. The input fluid connection is passed (guided) through the input fluid guide unit. A portion of the input fluid guide unit may belong to a wall of the measuring chamber. The input unit can be connected, preferably detachably connected, to the input fluid guide unit. The input fluid guide unit surrounds the valve, preferably completely. This implementation leads to a particularly space-saving configuration of the analyzer and requires less installation space than another possible arrangement of the valve. In addition, this implementation leads in many cases to a particularly robust configuration, and the risk of the valve being damaged or leaking due to external influences is reduced.

In a first application, a gas sample delivered by the subject is sucked once from the input unit into the measuring chamber, where it is analyzed by the sensor. As stated above, the invention facilitated ensuring that this gas sample consists predominantly of air from the subject's lungs, but includes only to a small extent air from the subject's upper airway and mouth. In this first application, the content of the substance in the blood of the subject is to be examined.

In a second application, two gas samples delivered by the same subject are sucked, one sample after the other, from the input unit into the measuring chamber, where the gas samples are analyzed by the sensor. In between, the measuring chamber is rinsed out (purged). In this second application, the analyzer is operated such that the first gas sample consists essentially of air from the subject's mouth, and the second gas sample consists essentially of air from the lungs. The first gas sample is used to determine whether the subject has recently ingested alcohol or some other substance that has not yet reached the subject's bloodstream. The second gas sample is used to examine the content of the substance in the subject's blood.

In a third application, a pre-sample is first aspirated into the measuring chamber, and the measuring chamber is rinsed out with this pre-sample without necessarily testing the pre-sample for the substance. The pre-sample can also come from the subject or from the environment.

In one embodiment, the analyzer is configured as a portable device and includes its own power supply unit, preferably a set of rechargeable batteries. The analyzer may also be configured as a stationary device and connected, or connectable, to a stationary power supply network.

In the following, the invention is described by means of embodiment examples. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view showing the mode of operation of an electrochemical sensor;

FIG. 2 is a perspective view, obliquely from above, of a first embodiment of the analyzer according to the invention;

FIG. 3 is a perspective view, vertically from above, of the analyzer of FIG. 2;

FIG. 4 is a cross-sectional view of the analyzer of FIG. 2 and FIG. 3;

FIG. 5 is another cross-sectional view of the analyzer of FIG. 2 and FIG. 3;

FIG. 6 is a perspective view of a segment of the analyzer according to the first embodiment comprising the sample inlet, the valve, the rod, the sensor, and the bellows, wherein the measuring chamber is omitted;

FIG. 7 is a cross-sectional view showing the segment of FIG. 6 with the sensor omitted;

FIG. 8 is a schematic cross-sectional view of the input fluid connection to the measuring chamber;

FIG. 9 is a cross-sectional view of a second embodiment of the analyzer according to the invention;

FIG. 10a is a sectional view through the analyzer with the rod perpendicular to the drawing planes taken along the plane A-A of FIG. 3;

FIG. 10b is a sectional view through the analyzer with the rod perpendicular to the drawing planes taken along the plane B-B of FIG. 3;

FIG. 10c is a sectional view through the analyzer with the rod perpendicular to the drawing planes taken along the plane C-C of FIG. 3.

### DESCRIPTION OF PREFERRED EMBODIMENTS

Referring to the drawings, in an embodiment example, the analyzer according to the invention is used to analyze a breath sample exhaled by a subject, in particular a human, for a predetermined substance, in particular breath alcohol. In the case of breath alcohol as the substance, a subject is to be analyzed to determine whether or not alcohol is present in his or her blood above a detection threshold. The subject inputs (delivers) a breath sample into a mouthpiece of the analyzer. If the subject has consumed alcohol and the alcohol in the blood has not yet completely decomposed, the breath sample delivered contains breath alcohol. A part of the delivered breath sample flows into a measuring chamber inside the analyzer. This portion is referred to below as the “measuring chamber sample”. A sensor in or on the measuring chamber checks whether or not this measuring chamber sample contains breath alcohol or any other predetermined substance. The invention can also be applied to another substance that may be present in the exhaled air of a subject or in any other gas that a subject may emit.

The sensor is capable of generating a signal that correlates with the amount and/or concentration of the given substance in the measuring chamber sample that is in the measuring chamber. Various suitable sensors are known from the prior art, for example electrochemical sensors, photo-optical sensors, photo-acoustic sensors, photo-ionization sensors, and heat tone sensors. Such a sensor can also be applied to the invention.

The analyzer derives the concentration of breath alcohol in the input breath sample from the measured amount or concentration of breath alcohol in the measuring chamber sample and the amount and/or volume of the measuring chamber sample. For example, the amount and/or volume of the measuring chamber sample is derived depending on the volume of the measuring chamber, which is known by the configuration of the analyzer, and/or by measuring the volume flow into the measuring chamber multiple times and integrating the measured values. If the substance is breath alcohol, a signal-processing evaluation unit of the analyzer or a spatially remote evaluation unit derives the current content of alcohol in the blood of the subject from the breath alcohol concentration in the breath sample.

As the breath sample passes through the mouthpiece, air first flows from the mouth, then from the upper respiratory tract (upper airway), and then air from the subject's lungs flows through the mouthpiece. To determine if the subject's blood contains alcohol, gas from that portion of the breath sample that originates from the lungs must be tested. Ideally, only gas originating from the subject's lungs will flow into the measuring chamber, and the measuring chamber sample will contain only air from the lungs, but no air from the mouth and upper airway. The following describes how this objective is achieved according to the invention.

The analyzer of the embodiment comprises an electrochemical sensor 12. By a “sensor” is meant a component that automatically generates a signal, preferably an electrical signal, wherein the generated signal is an indication of the amount and/or concentration of a predetermined substance in the measuring chamber sample. This measuring chamber sample is located in a measuring chamber, wherein the sensor is capable of analyzing this measuring chamber sample in the measuring chamber. An electrochemical sensor triggers a chemical reaction, wherein the chemical reaction depends on the amount and/or concentration of the substance to be analyzed and influences a measurable electrical detection quantity, for example the current intensity or the electrical voltage or the electrical charge or the electrical resistance of a component of the sensor.

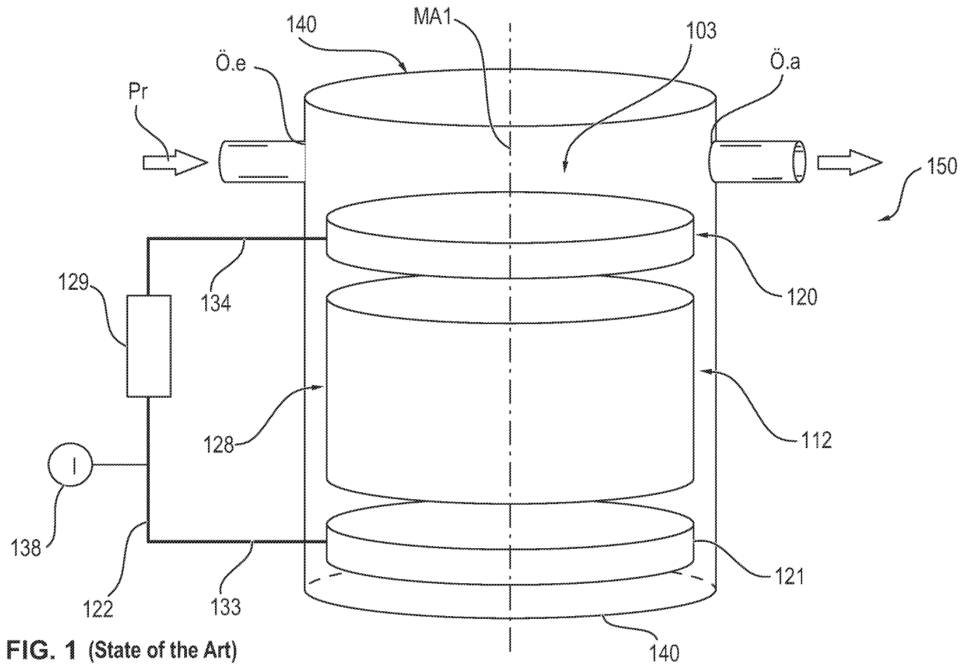

FIG. 1 shows schematically and by way of example the mode of operation of an electrochemical sensor 112 as known from the prior art. The representation of FIG. 1 is not necessarily true to scale. This electrochemical sensor 112 is capable of analyzing a measuring chamber sample Pr for breath alcohol and operates on the principle of a fuel cell with alcohol as the fuel. Features of such a sensor 112 can also be used for the analyzer according to the invention. In one embodiment, the analyzer according to the invention comprises the essential features of such an electrochemical sensor 112.

The reference number 150 in FIG. 1 denotes a sensor arrangement comprising an electrochemical sensor 112 and a wall 140 for a measuring chamber 103. The wall 140 surrounds the sensor 112 and the measuring chamber 103. In the form of implementation shown, both the wall 140 and the sensor 112 are rotationally symmetrical about the same central axis MA1. Of course, other geometric shapes are also possible.

The measuring chamber sample Pr to be analyzed, which in the embodiment comes from a breath sample A, flows through an opening Ö.e on the inlet side into the interior of the measuring chamber 103, e.g. by being exhaled or aspirated by a subject or by diffusing into the measuring chamber 103. In one embodiment, the measuring chamber sample Pr flows out of the measuring chamber 103 again through an outlet side opening Ö.a. Thanks to this embodiment, the sensor 112 can quickly examine several measuring chamber samples Pr in succession. It is also possible that there is no outlet-side opening Ö.a and the measuring chamber sample flows out of the measuring chamber 103 again through the inlet-side opening Ö.e.

The electrochemical sensor 112 comprises:
a measuring electrode 120, which is electrically contacted by a contacting wire 134,
a counter electrode 121, which is electrically contacted by a contacting wire 133,
an electrolyte 128 between the two electrodes 120 and 121,
a connecting wire 122 which electrically connects the two contacting wires 133 and 134 and in which an electrical measuring resistor 129 is arranged, and
a current intensity sensor (amperage meter) 138 that measures the intensity I of the current flowing through the connection wire 122.

Such an electrochemical sensor 112 is also referred to hereinafter as a membrane electrode electrolyte (MPEE) unit.

The electrolyte 128 is or comprises an electrically conductive medium, for example sulfuric acid or phosphoric acid or perchloric acid diluted with water. Ions can move in the electrolyte 128. Preferably, a porous membrane provides the electrolyte 128. The electrolyte 128 provides an ionically conductive connection between the measuring electrode 120 and the counter electrode 121, but electrically isolates the two electrodes 120 and 121 from each other.

The sensor 112 is configured such that the measuring chamber sample Pr reaches only the measurement electrode 120, but not the counter electrode 121. In the example shown, the measurement electrode 120 is located on a wall of the measuring chamber 3, and the wall 140 and the electrolyte 128 prevent a relevant amount of the measuring chamber sample Pr from reaching the counter electrode 121.

The two contact wires 133 and 134 are electrically conductive and made of a material that is not chemically attacked by the electrolyte 128, for example platinum or gold. The electrodes 120 and 121 are also made of a chemically resistant material, for example also platinum or gold. In many cases, the chemically resistant material of the electrodes 120, 121 additionally acts as a catalyst for a chemical reaction that depends on the substance to be detected and is used for measurement.

In one embodiment, the electrochemical sensor 112 operates on the principle of a fuel cell. The chemical reaction used for measurement includes the step of oxidizing the breath alcohol in the measuring chamber sample Pr in the measuring chamber 103. Ideally, the entire amount of breath alcohol in the measuring chamber sample Pr is oxidized.

As a result of the chemical reaction, an electric current flows between the measuring electrode 120 and the counter electrode 121 and thus through the connecting wire 122. The current intensity sensor 138 measures an indicator of the electric charge, i.e. of the total amount of electric current flowing through the connecting wire 122 (principle of coulometry). Generally, electric current flows until all combustible gas, in this case breath alcohol, is oxidized in the measuring chamber 103. For a given volume of measuring chamber sample Pr in measuring chamber 103, the more breath alcohol the measuring chamber sample Pr contains before oxidation, the higher the measured electric charge. The measured electric charge is therefore an indicator of the breath alcohol content in the measuring chamber sample Pr and thus of the alcohol content in the blood of the subject.

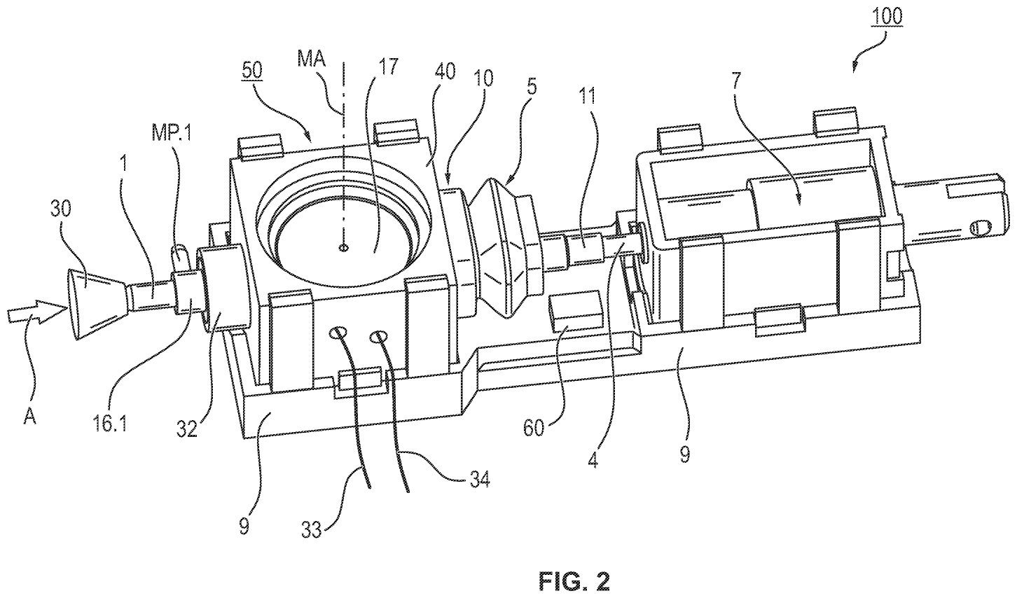

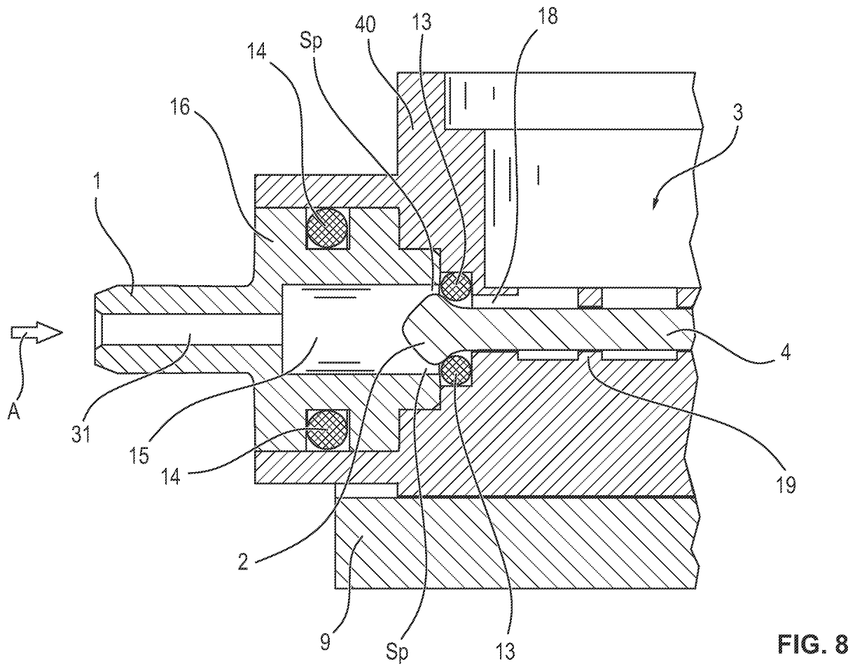

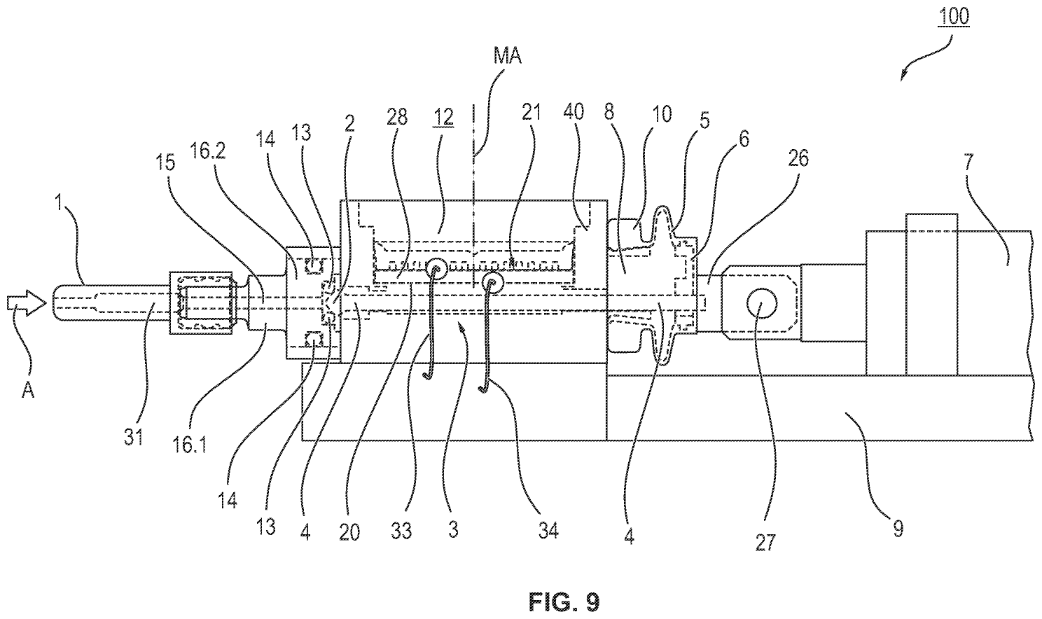

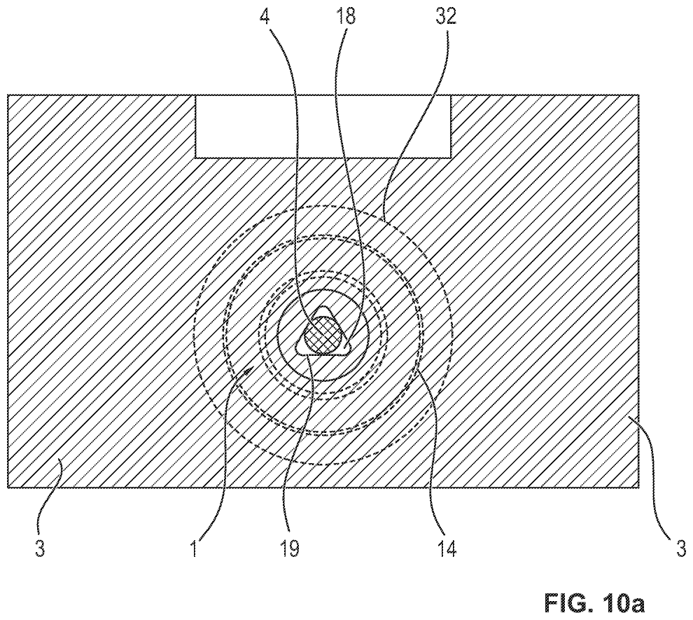

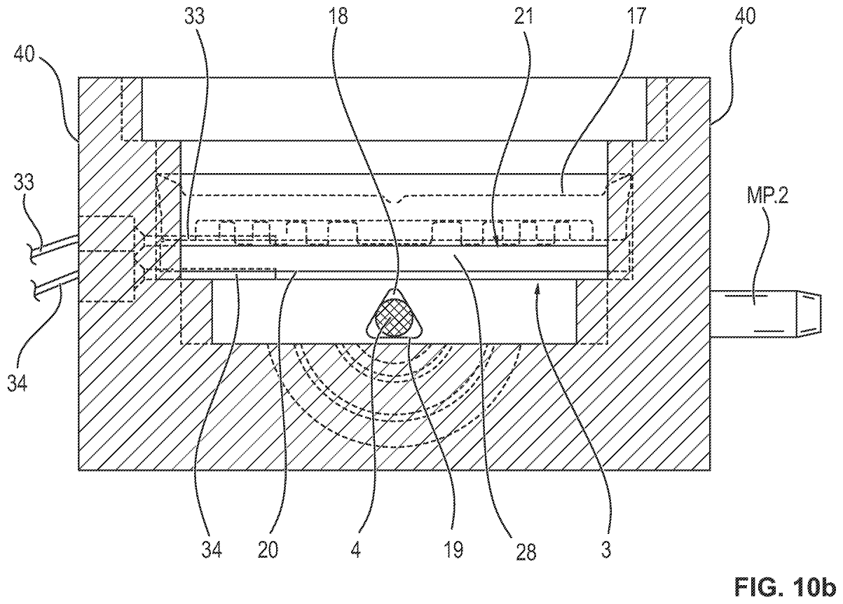

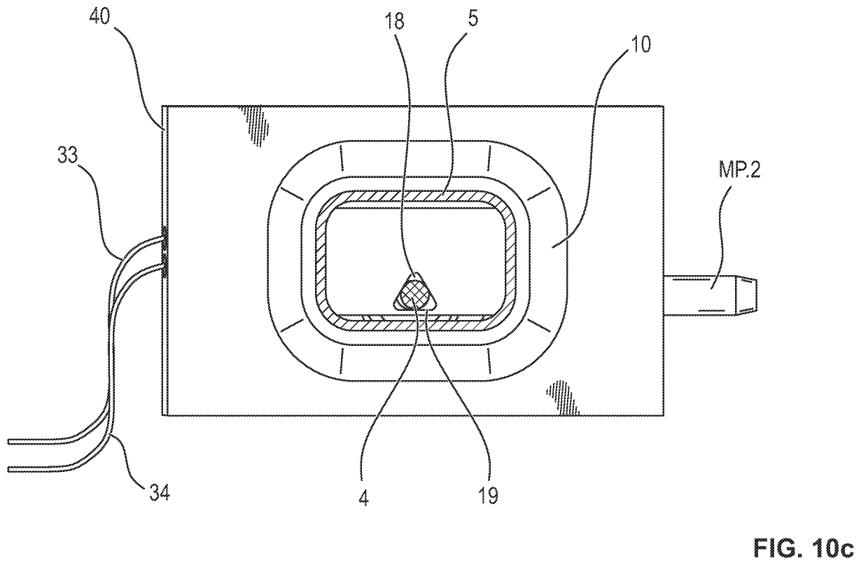

FIG. 2 to FIG. 7 show a first embodiment of an analyzer 100 according to the invention. FIG. 9 shows a second embodiment. FIG. 8 and FIGS. 10a, 10b and 10c are valid for both embodiments.

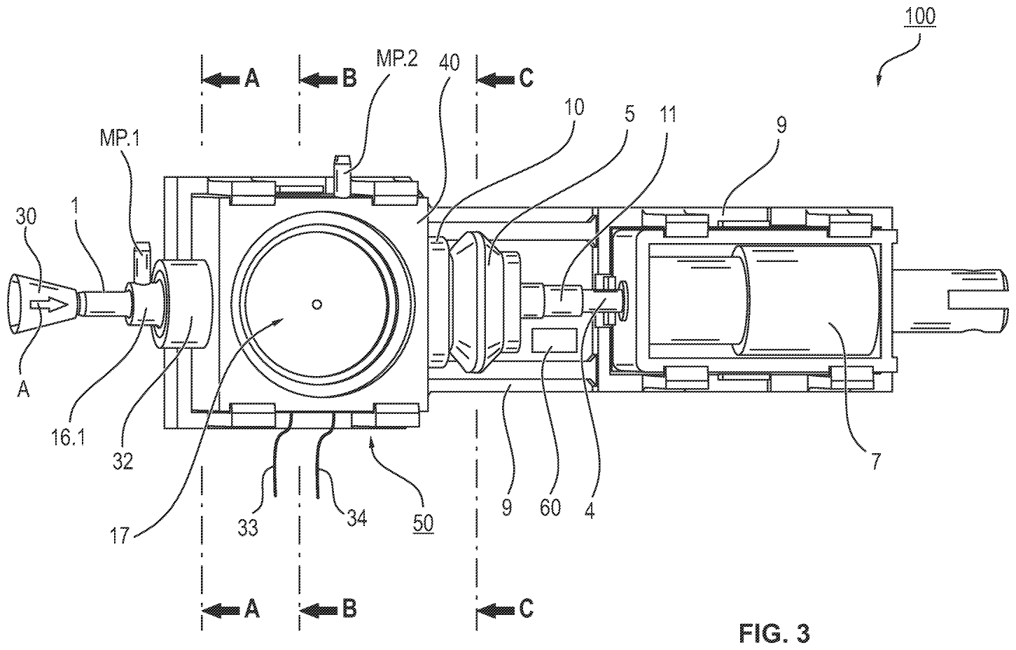

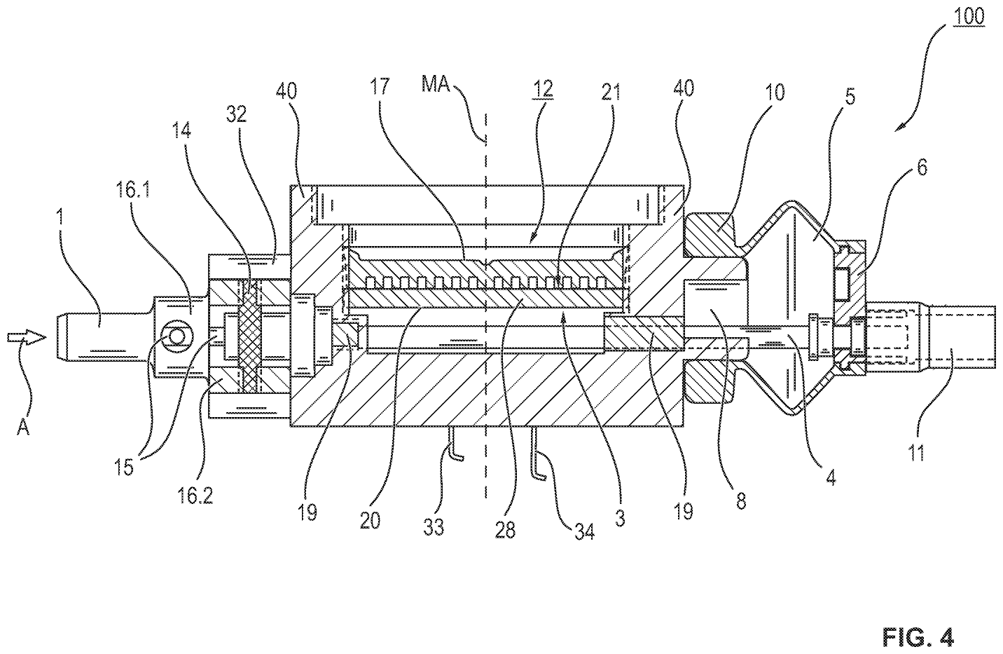

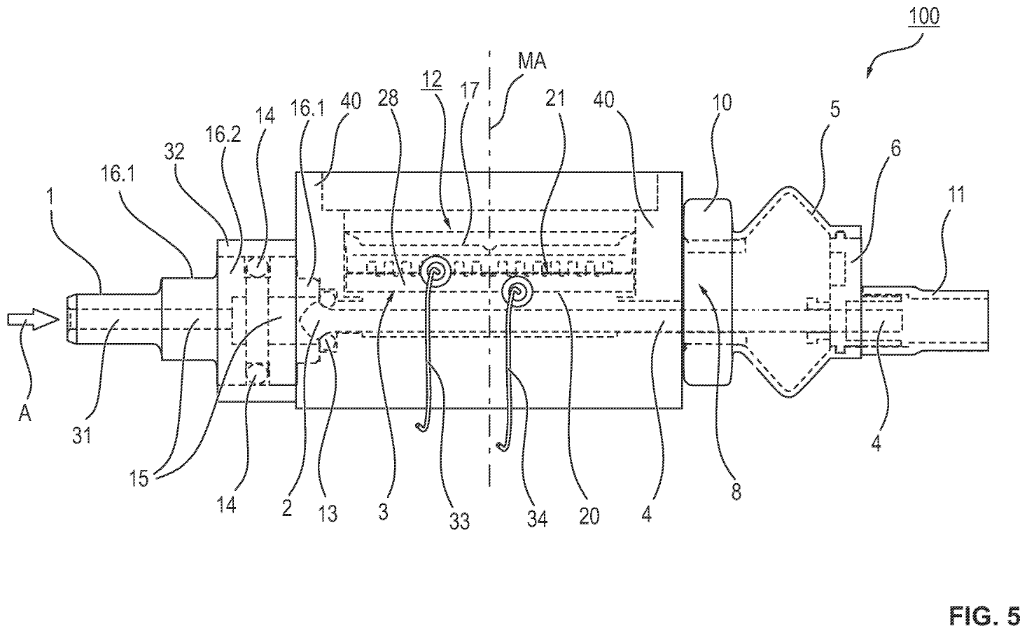

FIG. 2 and FIG. 3 show the analyzer 100 according to the first embodiment of the invention in a perspective view, FIG. 4 and FIG. 5 in two cross-sectional views.

The following additional components are mounted on a frame 9 of the analyzer 100:
a mouthpiece 30 shown only schematically,
a sample inlet 1,
a connecting piece 16, which consists of a smaller part 16.1 and a larger part 16.2, the two parts 16.1, 16.2 being firmly connected to each other,
a sensor arrangement 50 with a measuring chamber 3 and an electrochemical sensor 12, which electrochemical sensor 12 comprises a measuring electrode 20, which is electrically contacted by a contacting wire 34, a counter electrode 21, which is electrically contacted by a contacting wire 33, an electrolyte 28 between the two electrodes 20 and 21, a connecting wire which electrically connects the two contacting wires 33 and 34 and in which an electrical measuring resistor is arranged, and a current intensity sensor (amperage meter) that measures the intensity I of the current flowing through the connection wire, wherein the sensor arrangement 50 can be constructed, for example, comprising features of sensor arrangement 150 as shown in FIG. 1,
an upstream connector 32, which is attached to a wall 40 of the measuring chamber 3 upstream of the measuring chamber 3 and surrounds the part 16.2,
a connection piece 10 on the outflow side, which is attached to the wall 40 of the measuring chamber 3 downstream of the measuring chamber 3,
a linear sliding rod 4,
a connecting sleeve 11 through which the rod 4 is passed,
a bellows 5 acting as the suction chamber unit of the embodiment,
a plate 6 in the bellows 5, the plate 6 acting as the chamber modifying element,
an optional first measuring point MP.1 for a pressure measurement or volume flow measurement described below,
an optional second measuring point MP.2 for another pressure measurement or for this volume flow measurement,
an actuator that can move an object in two opposite directions and whose function is described below,
a schematically shown control unit 60, which receives a signal from the sensor arrangement 50 and from a volume flow sensor or a pressure sensor, respectively, and is configured to control the actuator, and
a power supply unit not shown, for example at least one battery (accumulator), which supplies the actuator with electrical energy.

The designations “front” and “back” and “upstream” and “downstream” refer to the direction of flow of a gas from sample inlet 1 to bellows 5, i.e. from left to right in FIG. 2 to FIG. 9.

The mouthpiece 30 is attachable to the sample inlet 1 and removable from the sample inlet 1. In one embodiment, the attached mouthpiece 30 surrounds the sample inlet 1. The mouthpiece 30 has the shape of a funnel, whereby this funnel tapers towards the sample inlet 1 when the mouthpiece 30 is attached. Thanks to this funnel shape, an overpressure is created inside the mouthpiece 30 when a subject supplies (delivers) a breath sample A.

The mouthpiece 30 belongs to the input unit of the embodiment, the sample inlet 1 and the connecting piece 16 to the input fluid guide unit. An input fluid connection described below passes through the input fluid guide unit and is capable of connecting the mouthpiece 30 to the measuring chamber 3.

The mouthpiece 30 has an opening through which the breath sample can flow to the sample inlet 1. Preferably, further openings (not shown) are formed into the mouthpiece 30. Breathing air can escape into the environment through these further openings, in particular if excess pressure has developed in the mouthpiece 30. This reduces the risk that, in the event of excess pressure in the mouthpiece 30, part of the breath sample A will return to the subject. A mouthpiece with such openings is described by way of example in DE 10 2017 008 008 A1 (corresponding U.S. Pat. No. 11,474,096 (B2) is incorporated herein by reference).

The measuring chamber 3 is surrounded by the wall 40 and a cover plate 17. The sensor 12 is arranged under the cover plate 17. In the shown embodiment example, the wall 40 of the sensor arrangement 50 has an outer contour in the form of a cuboid and an inner contour in the form of a cylinder. Other geometric shapes are also possible. The sensor 12 and the measuring chamber 3 are rotationally symmetrical about the same central axis MA. This central axis MA is perpendicular to the drawing plane of FIG. 3 and lies in the drawing planes of FIGS. 4 and FIG. 5.

In the example shown, the actuator comprises a solenoid 7 and a reset unit in the form of a spring which is supported on the frame 9 and connected to the solenoid 7. The power supply unit, which is not shown, is electrically connected to the solenoid 7. Other configurations of an actuator are also possible, for example an electric motor or a piston-cylinder unit. Even a manual drive may be provided.

Also shown in the cross-sectional views of FIG. 4 and FIG. 5 are the following components of the analyzer 100:
a valve with a closure part and a closure part seat 13,
a cavity 31 in the form of a tube in the sample inlet 1,
a cavity 15 in the form of a tube inside the connector 16,
the plate 6 in the bellows 5, the plate 6 being firmly connected to the rod 4,
a guide unit 19 which guides the rod 4 linearly along the longitudinal axis of the rod 4 and prevents lateral movement or rotation or canting of the rod 4,
a section through the sensor 12 with the measuring electrode 20, the counter electrode 21 and the electrolyte 28 and
a section through the wall 40 of the measuring chamber 3.

In addition, FIG. 4 and FIG. 5 show how the linear sliding rod 4 and the connecting sleeve 11 connect the sealing cone (sealing part) 2 to the solenoid 7.

In the embodiment example, the sealing part 2 has the shape of a sealing cone, and the sealing part seat 13 has the shape of a sealing ring, which is preferably elastic. The diameter of the sealing cone 2 is preferably larger than the diameter of the rod 4, making it possible to make the diameter of the sealing cone 2 as large as possible and the diameter of the rod 4 as small as possible. In any position of the rod 4, the sealing cone 2 is located in the cavity 15. A circumferential gap Sp occurs between the sealing cone 2 and the inner wall of the cavity 15, see FIG. 5 and FIG. 8.

The sealing element seat (sealing ring) 13 surrounds that end of the rod 4 which is adjacent to the sealing cone 2 and is recessed in a recess in the wall 40. The rod 4 passes through the measuring chamber 3, cf. FIG. 4 and FIG. 5. In the embodiment shown, the longitudinal axis of the rod 4 is perpendicular to the central axis MA of the cylindrical measuring chamber 3 and lies in the drawing planes of FIG. 3, FIG. 4 and FIG. 5. In one embodiment, at least one optional mixing element (not shown) in the form of a flat component is fixedly mounted on the rod 4. The mixing element or each mixing element is located inside the measuring chamber 3 in any position of the rod 4.

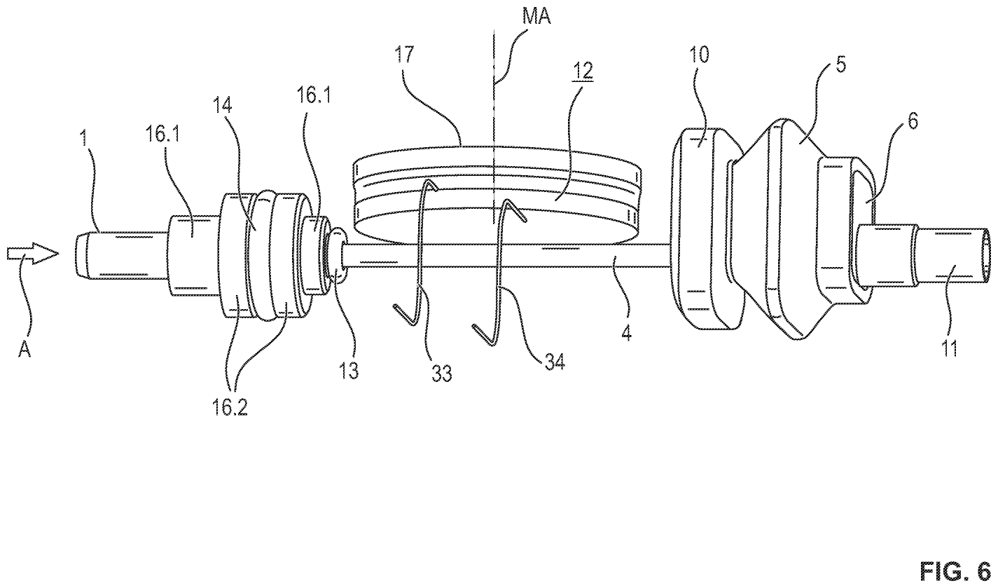

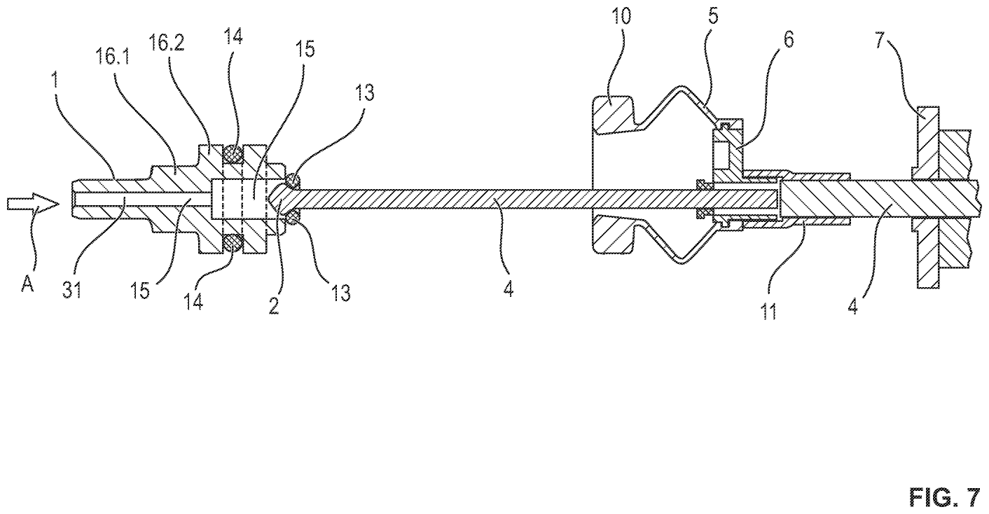

FIG. 6 and FIG. 7 show the sample inlet 1, the valve 2, 13 and the input fluid connection between the sample inlet 1 and the measuring chamber 3 in a perspective view and in a cross-sectional view, respectively. FIG. 8 illustrates the input fluid connection between the sample inlet 1 and the measuring chamber 3 in a schematic cross-sectional view and in a slightly different implementation form from FIG. 2 to FIG. 7.

In the embodiments shown, the measuring chamber 3 is in fluid connection with the mouthpiece 30 exclusively via the input fluid connection, and only when the valve 2, 13 is fully or at least partially open.

Two alternative embodiments are also possible, neither of which are shown:
In the first alternative embodiment, the mouthpiece 30 is in fluid connection with the environment via a separate output fluid connection. Preferably, this output fluid connection branches off from the input fluid connection upstream of the valve 2, 13. Preferably, this output fluid connection is closed when the valve 2, 13 is open and is open when the valve 2, 13 is closed. When the output fluid connection is open, breathing air that has been input into the mouthpiece 30 flows through the output fluid connection into the environment, particularly when the valve 2, 13 is closed. This embodiment reduces the risk of gas that the subject has input into the mouthpiece 30 flowing back to the subject.
In the second alternative embodiment, the measuring chamber 3 is in fluid connection with the environment via an outlet fluid connection. The measuring chamber 3 can be flushed out through this outlet fluid connection. Preferably, a valve is arranged in this outlet fluid connection, which is only opened when gas is to be removed from the measuring chamber 3. This embodiment avoids that gas is conducted or conveyed from the measuring chamber 3 into the mouthpiece 30 during flushing of the measuring chamber 3.

In the illustrations from FIG. 2 to FIG. 7, the bellows 5 is shown in a maximum volume state. The rod 4 couples this state in the first embodiment according to FIG. 2 to FIG. 7 with a state in which the valve 2, 13 is in the closing end position.

FIG. 9 shows the bellows 5 in a minimum volume state. In the second embodiment according to FIG. 9, the rod 4 couples this state with the state in which the valve 2, 13 is in the closing end position. The same reference signs have the same meanings as in FIG. 2 to FIG. 7.

FIG. 9 also shows the following components:
a connecting element 26 between the plate 6 and the solenoid 7 and
a bolt (pin) passing through a recess in the connecting element 26 and through a recess in a rod of the solenoid 7.
   The wall structure 40 and rod 4 can be tilted or have an angular variation (sensor 12 can rotate) relative to the solenoid 7 about the longitudinal axis of the bolt 27, thanks to the bolt 27.

FIGS. 10a, 10b and 10c shows three sections through the analyzer 100, with the axis of the rod 4 perpendicular to the respective drawing plane in each section and the viewing direction of solenoids 7 directed towards the sample inlet 1. These three sections apply to both embodiments of the analyzer 100. FIG. 10a shows a section in the plane A-A of FIG. 3, FIG. 10b shows a section in the plane B-B of FIG. 3 and FIG. 10c shows a section in the plane C-C of FIG. 3.

The following other components of the analyzer 100 are shown in FIG. 6, FIG. 7, and/or FIG. 8:
a tubular recess 18 surrounding the rod 4 and has an approximately triangular cross-sectional area,
a guide unit 19 for the rod 4 and
another sealing ring 14 around the tube 16.

The cavities 31 and 15 together form a tube that continues into the recess 18. In the embodiment example, the tube 31, 15, the gap Sp and the recess 18 together provide the input fluid connection between the sample inlet 1 and the measuring chamber 3. The sealing cone 2 is movable back and forth between a closing end position, in which the input fluid connection 31, 15, Sp, 18 is interrupted, and a releasing end position, in which the input fluid connection 31, 15, Sp, 18 is released. In the closing end position, shown in the figures, the sealing cone 2 is in fluid-tight contact with the closing element seat (sealing ring) 13. By moving the sealing cone 2 away from the closure element seat 13 and towards the sample inlet 1 (first embodiment) or away from the sample inlet 1 (second embodiment), the sealing cone 2 is moved linearly into the releasing end position.

When the sealing cone 2 is in the releasing end position or in an intermediate position between the releasing and closing end positions, an input fluid connection 31, 15, Sp, 18 is established between the mouthpiece 30 and the measuring chamber 3. This input fluid connection 31, 15, Sp, 18 passes through the following components:
the tube 31,
the cavity 15,
the gap Sp between the sealing cone 2 and the inner wall of the cavity 15,
the inner space enclosed by the sealing ring 13 and
the recess 18 around the rod 4.

When the sealing cone 2 is in the closing end position, i.e. rests against the sealing ring 13, this input fluid connection 31, 15, Sp, 18 is interrupted.

The rod 4 and the connecting sleeve 11 connect the sealing cone 2 with the solenoid 7. The actuator with the solenoid 7 and the spring (not shown) can move the rod 4 linearly in both directions and thus move the sealing cone 2 back and forth between the closing end position and the releasing end position. The rod 4 is guided through the connecting sleeve 11.

The rod 4, the connecting sleeve 11 and the plate 6 are mechanically connected to each other in such a way that they cannot move relative to each other. The connecting sleeve 11 transmits a movement of the rod 4 to the plate 6. Together with the rod 4, the linear solenoid 7 can also move the connecting sleeve 11 and thus the plate 6 linearly.

The bellows 5 is mechanically connected to the wall 40 on the side facing the sample inlet 1. The connecting piece 10 surrounds the bellows 5. The plate 6 limits the bellows 5 on the opposite side. A linear movement of the plate 6 towards the sample inlet 1 compresses the bellows 5 and transfers the bellows 5 into the minimum volume state. A linear movement of the plate 6 in the opposite direction pulls the bellows 5 apart and transfers the bellows 5 into the maximum volume state. The bellows 5 is in suction-fluid connection 8 with the measuring chamber 3.

The solenoid 7, the spring, the bellows 5 and the plate 6 together form a displacement pump. Instead of a solenoid 7, the analyzer 100 can also have another controllable actuator, whereby this actuator can move the rod 4 in both directions and hold it in an end position. A manual actuator may also be provided.

Instead of a bellows 5 and a plate 6, a piston-cylinder unit (not shown) can also be used, whereby the actuator 7 is capable of moving the piston relative to the cylinder. It is also possible that the other actuator just mentioned is capable of moving the piston of a piston-cylinder unit. It is also possible that an electric motor is capable of moving the rod 4 linearly in both directions.

The optional first measuring point MP.1 is in fluid connection with the sample inlet 1 or with the cavity 15 and thus in fluid connection with the input fluid connection 31, 15, Sp, 18 just described, which connects the mouthpiece 30 to the measuring chamber 3. Therefore, the first measuring point MP.1 is in fluid connection with the mouthpiece 30 even when the valve 2, 13 is closed. It is also possible that the first measuring point MP.1 is in fluid connection with the recess 18. The optional second measuring point MP.2 is arranged between the measuring chamber 3 and the solenoid 7 and is in fluid connection with the measuring chamber 3.

Two pressure sensors, which are not shown, measure the pressure at the first measuring point MP.1 and at the second measuring point MP.2, respectively. At each sampling point of a sequence of sampling points, two pressure measurements are performed, respectively. Preferably, these two pressure sensors measure the respective pressure difference with respect to the ambient pressure in the environment of the analyzer 100.

In one embodiment, the measured values of that pressure sensor which is connected to the first measuring point MP.1 are used to determine approximately the volume flow into the mouthpiece 30 and thus the volume of that amount of breath sample A which has been input into the mouthpiece 30 so far. At least when the valve 2, 13 is closed, this inputted amount of breath sample A essentially causes the pressure inside the funnel-shaped mouthpiece 30 to increase. The slots in the mouthpiece 30 can only partially relieve this excess pressure. The information about the volume of the quantity delivered so far can be used to trigger the operation of opening the valve 2, 13. As already explained, only air from the subject's lungs should enter the measuring chamber 3, but not air from his or her mouth and upper airways. How this desired effect is achieved is described in more detail below

In one embodiment, the measured values of the pressure sensor connected to the second measuring point MP.2 are used to measure the time course of the pressure in the measuring chamber 3. From this time course of the pressure as well as the volume of the measuring chamber 3, which is known by the configuration of the analyzer 100, an estimated value for the amount of the measuring chamber sample can be derived.

In a further embodiment, which can be combined with the two embodiments just described, a volume flow sensor not shown derives the difference between the measured pressure at the first measuring point MP.1 and the measured pressure at the second measuring point MP.2. This pressure difference is an indicator of the current volume flow from and into the measuring chamber 3. Optionally, it is also automatically checked whether the valve 2, 13 is tight, i.e. whether it actually interrupts the input fluid connection in the closing position.

FIG. 8 illustrates how the rod 4 (shown dashed in FIG. 9) is guided. In the example shown, the recess 18 has a triangular cross-section so that a breath sample A can flow past the rod 4 to the measuring chamber 3. Furthermore, the rod 4 is guided linearly by the guide unit 19. Thanks to this guidance, the rod 4 can only move linearly in two directions parallel to its own longitudinal axis, but cannot move laterally or tilt.

FIGS. 10a, 10b and 10c show the triangular cross-sectional area of cavity 18.

The following describes how the analyzer 100 collects and analyzes a breath sample A.

Before use, the analyzer 100 is in an idle state. No mouthpiece 30 is placed on the sample inlet 1. A mechanical or pneumatic spring (not shown) of the actuator is supported on the frame 9 and holds the rod 4 in a position in which the rod 4 has the maximum possible distance from the sample inlet 1 in the first embodiment, and in a position with minimum possible distance from the sample inlet 1 in the second embodiment. The solenoid 7 is deactivated, i.e. no current flows through it. Thanks to the spring, the plate 6 pulls the bellows 5 apart in the first embodiment according to FIG. 2 to FIG. 7, so that the bellows 5 has the maximum volume. In the second embodiment, the plate 6 compresses the bellows 5 in the rest state thanks to the spring, so that the bellows 5 has the minimum volume.

The sealing cone 2 is in the sealing end position prior to use, and the valve 2, 13 closes the input fluid connection 15, 18 between the sample inlet 1 and the measuring chamber 3. Therefore, the measuring chamber 3 is not in fluid connection with the environment. Particles, substances and other environmental influences can therefore not affect the electrochemical sensor 12 while the analyzer 100 is at rest, and conversely there is little risk of components of the electrolyte 28 leaving the electrochemical sensor 12 or even the measuring chamber 3, for example due to evaporation.

In one embodiment, the mouthpiece 30 is used to input a single breath sample A and is then discarded. In another embodiment, the mouthpiece 30 is disinfected after the input of a breath sample A and then reused.

In either embodiment, the mouthpiece 30 is not connected to the remainder of the analyzer 100 until a deployment of the analyzer 100 begins and a subject inputs a breath sample A. Preferably, the event of the mouthpiece 30 being placed on the sample inlet 1 triggers the step of transferring the analyzer 100 from an idle state to a deployed state. For example, a contact switch detects the event that the mouthpiece 30 has been placed on the sample inlet 1.

During an operation, a subject inputs (delivers) a breath sample A into the attached mouthpiece 30. This breath sample A initially contains exhaled air from the mouth and upper respiratory tract and then exhaled air from the subject's lungs. Ideally, the analyzer 100 examines only exhaled air from the lungs. Therefore, the valve 2, 13 initially remains closed even if the subject has already begun to input a breath sample A into the mouthpiece 30. As mentioned above, the mouthpiece 30 preferably includes several other openings so that the input breath sample A can fully exit into the environment as long as the valve 2, 13 is closed and is not blown into the subject's face.

In the deployment state, the analyzer 100 automatically detects the occurrence of a predetermined opening event.

Before the opening event has occurred, the valve 2, 13 is closed and the input air escapes back out of the mouthpiece 30 through the slots or through the output fluid connection, thus ensuring that air actually flows from the subject's lungs into the measuring chamber 3 and, in particular, that no significant amount of air escapes from the mouth and upper airways.

For example, this opening event has occurred when a predetermined period of time has elapsed since the step of putting on the mouthpiece 30. Or, the opening event has occurred when a predetermined amount of breath sample A has been input into the mouthpiece 30 since the step of putting on the mouthpiece 30, or when the subject has completed the step of inputting a breath sample A. In the second alternative, a sensor measures an indicator of the volume of gas delivered into the mouthpiece 30 or the volume flow of gas into the mouthpiece 30, as described in more detail below.

Detection that the opening event has occurred triggers the following steps in the first embodiment shown in FIG. 2 through FIG. 7:
A circuit is closed and electric current activates the solenoid 7.
The activated solenoid 7 pushes the rod 4 towards the sample inlet 1 against the force of the spring.
Moving the rod 4 towards the sample inlet 1 causes the sealing cone 2 to be pushed away from the sealing ring (closing element seat) 13 and towards the sample inlet 1. This causes the valve 2, 13 to open, namely to move it into the releasing end position. This releases the input fluid connection described above between the input unit (the mouthpiece 30 and the sample inlet 1) and the measuring chamber 3.
Moving the rod 4 also causes the plate 6 to compress the bellows 5.
Because the bellows 5 is compressed, gas flows from the bellows 5 through the inlet fluid connection 8 and into the measuring chamber 3. This pushes existing gas from the measuring chamber 3 through the inlet fluid connection 18, 15 and out of the analyzer 100 through the sample inlet 1. As a result, the measuring chamber 3 is purged. The purged gas in the measuring chamber 3 may be from a previous input.
The pressure caused by compressing the bellows 5 is much greater than the pressure caused by inputting the breath sample A into the mouthpiece 30. Therefore, no significant amount of the input breath sample A flows into the input fluid connection while the volume of the bellows 5 is still being reduced.
Once the bellows 5 is fully compressed, no more gas is forced out of the measuring chamber 3 through the input fluid connection. The valve 2, 13 is open and gas can be sucked or flow from the mouthpiece 30 through the input fluid connection into the measuring chamber 3.

As soon as a closing event is detected, the following steps are triggered:
The rod 4 is again pushed away from the sample inlet 1 until the valve body 2 reaches the closure element seat 13. For example, solenoid 7 is de-energized again and the spring moves rod 4 away from sample inlet 1.
As soon as the valve body 2 reaches the closing element seat 13, the input fluid connection 31, 15, Sp, 18 is closed again, and the measuring chamber 3 is separated fluid-tightly from the mouthpiece 30 and from the environment.
Moving the rod 4 away from the sample inlet 1 also causes the plate 6 to pull the bellows 5 apart. Pulling the bellows 5 apart creates a negative pressure. The negative pressure causes gas to be drawn from the mouthpiece 30 through the cavity 31 in the sample inlet 1 and the input fluid connection 31, 15, Sp, 18 in the connector 16 into the measuring chamber 3. The amount of gas drawn into the measuring chamber 3 by this negative pressure belongs to the measuring chamber sample.
Moving the rod 4 further causes the optional mixing element or each optional mixing element on the rod 4 to move through the measuring chamber 3, thereby mixing the gas in the measuring chamber 3 to some degree.
As soon as the plate 6 has completely pulled the bellows 5 apart, the bellows 5 has reached its maximum volume. The valve 2, 13 has again reached the closing end position.

The electrochemical sensor 12 analyzes the gas sample in the measuring chamber 3, for example as described with reference to FIG. 1. Here, the measuring electrode 20 oxidizes the breath alcohol present in the measuring chamber 3. The measuring chamber sample is in the measuring chamber 3 until the rod 4 is pushed towards the sample inlet 1 again, thereby compressing the bellows 5. The time interval between
the event that the bellows 5 is fully extended and the valve 2, 13 is closed, and
the event that is started to compress the bellows 5 again, is available to the electrochemical sensor 12 for analysis, in particular for oxidation, of the measuring chamber sample. During this period, the valve 2, 13 is closed. As a rule, this time period is sufficient to completely oxidize alcohol in the measuring chamber sample.

The second embodiment according to FIG. 9 has the following deviations from the first embodiment according to FIG. 2 to FIG. 7:
The sealing ring 13 is located upstream of the sealing cone 2, while in the first embodiment the sealing ring 13 is located downstream of the sealing cone 2.
At rest, the spring, which is not shown, holds the rod 4 in a position in which the rod 4 is the smallest possible distance from the sample inlet 30.
The activated solenoid 7 pulls the rod 4 away from the sample inlet 1 against the force of the spring.
Moving the rod 4 away from the sample inlet 1 causes the sealing cone 2 to move away from the sealing ring 13 and toward the measuring chamber 3, thereby opening the valve 2, 13.
In addition, moving the rod 4 away from the sample inlet 1 causes the plate 6 to pull the bellows 5 apart.
Because the bellows 5 is pulled apart, gas flows through the input fluid connection 31, 15, Sp, 18 into the measuring chamber 3. The gas that flows into the measuring chamber 3 in this manner belongs to the measuring chamber sample.
The closing event triggers the step of pushing the rod 4 back towards the sample inlet 1, for example by the spring.
The sealing cone 2 is moved away from the measuring chamber 3 and towards the sealing ring 13. As soon as the sealing cone 2 has reached the sealing ring 13, the valve 2, 13 is closed again.
Moving the rod 4 toward the sample inlet 1 also causes the plate 6 to push the bellows 5 back together. The pushing together of the bellows 5 causes an overpressure. This overpressure causes gas, and thus the measuring chamber sample, to be pushed out of the bellows 5 through the suction fluid connection 8 and into the measuring chamber 3. This causes gas to be pushed out of the measuring chamber 3 through the input fluid connection 15, Sp, 18 and through the cavity 31 into the sample inlet 1. This flushes the measuring chamber 3.

In the second embodiment according to FIG. 9, the following time period is available for the sensor 12 to analyze the measuring chamber sample: the time period between
the event that the bellows 5 is fully extended, and
the event that the collapsing of the bellows 5 is started again.
   During this period, the valve 2, 13 is fully open.

In order for the electrochemical sensor 12 to reliably analyze a gas and thus also the measuring chamber sample in the measuring chamber 3 for the presence of breath alcohol and to measure the amount or concentration of breath alcohol in the measuring chamber 3, it should be known at least approximately what amount (mass) of the breath sample A is in the measuring chamber 3 during the analysis, thus what amount the measuring chamber sample has. Based on the design of the analyzer 100, the volume of the measuring chamber 3 is known. The difference between the maximum volume and the minimum volume of the bellows 5 is also known by design. Ideally, only air from the subject's lungs flows into the measuring chamber 3, but no air from the mouth and upper airways, so that the measuring chamber sample consists only of air from the lungs.

In both of the above-described embodiments, gas is drawn into the measuring chamber 3 by pulling the bellows 5 apart, thereby changing it from the minimum volume state to the maximum volume state. The difference between the maximum volume and the minimum volume of the bellows 5 is in many cases equal to the volume of breathing air drawn into the measuring chamber 3 from the mouthpiece 30.

In addition, during a period of time when the valve 2, 13 is or will be open and at the same time the bellows 5 is not moved, gas may flow into the measuring chamber 3, for example because the subject continues to exhale further or by diffusion. In many cases, however, the amount of gas that flows into the measuring chamber 3 when the bellows 5 is not moving can be neglected.

In one embodiment, a pressure sensor that is in fluid connection with the measuring position MP.1 measures the time course of an indicator of the overpressure in the mouthpiece 30 relative to the ambient pressure. From this time course of pressure, it is possible in some cases to derive which volume of breathing gas flows into the measuring chamber 3 in a period of time in which the valve 2, 13 is or becomes open and at the same time the bellows 5 is not moved.

In one embodiment, a volume flow sensor measures the difference between the pressures at the two measuring points MP.1 and MP.2 and determines the volume flow from this. By integrating over a certain period of time, the volume flowing through the input fluid connection 31, 15, Sp, 18 into the measuring chamber 3 during this period of time is derived from the volume flow. This time span is, for example, equal to the time span in which the valve 2, 13 is open and at the same time the bellows 5 is not moved. Optionally, the measuring time period additionally comprises the time period in which the valve body 2 is moved. It is also possible that this measurement time period also includes the time period in which the bellows 5 is pulled apart, so that the volume flow sensor is also used to measure which volume is sucked into the measuring chamber 3.

To deliver a valid breath sample A, the subject must exhale into the mouthpiece 30 during the procedure just described and thereby deliver the breath sample A at least until the bellows 5 is fully extended. If the subject cancels the delivery of the breath sample A before then, a corresponding message is preferably output in a form that can be perceived by a human. Preferably, the patient can then deliver another breath sample A.

Various embodiments of how the opening event may be determined are described below. As mentioned earlier, the valve 2, 13 is then started to move to the releasing end position when the opening event is detected. Ideally, the opening event has occurred when air from the subject's lungs has reached the mouthpiece 30.
In one embodiment, the opening event has occurred when a predetermined period of time has elapsed since the mouthpiece 30 was placed on the mouthpiece.
In another embodiment, an approximate measurement is made of the amount of exhaled air the subject has input into the mouthpiece 30 since the mouthpiece 30 was put in place. As explained above, in one embodiment, a pressure sensor in fluid connection with the first measurement point MP.1 measures an indicator of the positive pressure in the mouthpiece 30 relative to the ambient pressure several times in succession. An estimated value for the previously input volume is derived at least once from the measured values for the pressure difference. When the volume delivered so far reaches a predetermined volume threshold, the opening event has occurred. This volume threshold is preferably equal to the average volume of the mouth and upper airway of an adult.
Another embodiment can be used, in particular, in conjunction with the output fluid connection described above and not shown, namely when the output fluid connection connects the mouthpiece 30 to the environment. As long as the valve 2, 13 is closed, the respiratory air that the subject has input into the mouthpiece 30 is passed through the output fluid connection to the environment. The volume flow sensor described above with the two measuring positions MP.1 and MP.2 measures the volume flow through the input fluid connection 31, 15, Sp, 18 and the output fluid connection. The volume delivered so far is derived from the measured volume flow. As soon as the volume delivered so far reaches the volume threshold, the opening event has occurred.

The step of starting the movement of the valve 2, 13 back into the closing end position is triggered by the closing event. Various configurations are possible as to when the closing event has occurred:
In one embodiment, the closing event occurs as soon as the valve 2, 13 has reached the releasing end position. The valve 2, 13 thus remains in the releasing end position for only a very short period of time, ideally for only one instant. Gas is drawn into the measuring chamber 3 exclusively by the bellows 5 being pulled apart, i.e. being transferred from the with minimum volume state into the maximum volume state. The quantity, for example the mass, of the measuring chamber sample that enters the measuring chamber 3 is determined by the difference between the maximum volume and the minimum volume of the bellows 5.
In another embodiment, it is automatically determined when the chemical reaction in the measuring chamber 3 has ended. At the end of the chemical reaction, all breath alcohol in the measuring chamber 3 is oxidized. To determine this event, the time course of the signal generated by the sensor 12 is determined. If the signal from the sensor 12 remains approximately constant, the chemical process is complete. The completion of the chemical process acts as the closing event.

This other embodiment can be used in particular in conjunction with the second embodiment (FIG. 9). This is because in the second embodiment, the closing event causes the bellows 5 to be compressed and the measuring chamber sample to be forced out of the measuring chamber 3, thereby flushing the measuring chamber 3. Thus, by this time, the analysis of the measuring chamber sample must be completed. Before this, the input fluid connection is established.

In the embodiments described so far, breathing air, which ideally comes only from the subject's lungs, enters the measuring chamber 3 and functions there as the measuring chamber sample to be analyzed. It is possible that a preliminary sample is additionally drawn into the measuring chamber 3 in advance and expelled from the measuring chamber 3 again before air from the subject's lungs enters the measuring chamber 3. The bellows 5 is thus pulled apart and compressed again twice in order to test the same subject for alcohol. As a rule, this presample consists predominantly of air originating from the mouth and/or the upper respiratory tract of the subject. The measuring chamber 3 is rinsed out with the aid of the preliminary sample. In one embodiment, the level of breath alcohol in the mouth and/or in the upper respiratory tract of the subject is also determined at least approximately. In another embodiment, the presample is used to bring the electrodes 20, 21 of the sensor 12 to the temperature of the breath sample A. Typically, the breath sample A has a higher temperature than the ambient air. This embodiment increases the reliability of the measurement result in some cases. The two embodiments just described can be combined.

The bellows 5 is in fluid connection with the measuring chamber 3 through the inlet fluid connection 8. The action of compressing the bellows 5 causes gas to be forced into the measuring chamber 3 through the inlet fluid connection 8, thereby purging the measuring chamber 3. In the embodiments described thus far and shown in the figures, the gas that is forced out of the measuring chamber 3 is forced into the mouthpiece 30 through the input fluid connection 31, 15, Sp, 18.

In a different and not shown embodiment, the analyzer additionally comprises an outlet fluid connection. The measuring chamber 3 is in fluid connection with the environment via this outlet fluid connection. A three-way valve can optionally be brought into one of the following three positions:
to an inlet position where the three-way valve clears the inlet fluid connection 31, 31, 15, Sp, 18 while blocking the outlet fluid connection,
to an outlet position in which the three-way valve releases the outlet fluid connection while blocking the input fluid connection 31, 31, 15, Sp, 18, and
optionally to a blocking position in which the three-way valve blocks both fluid connections.

During the process of pulling the bellows 5 apart, the three-way valve is in the inlet position so that gas can flow through the inlet fluid connection 31, 15, Sp, 18 into the measuring chamber 3. During the process of compressing the bellows 5, the three-way valve is in the outlet position so that gas can flow out of the measuring chamber 3 through the outlet fluid connection. This embodiment results in gas being expelled into the environment rather than into the mouthpiece 3 when the measuring chamber 3 is purged. Preferably, the three-way valve is in the closed position while the bellows 5 is not moved.

In one embodiment, the opening event causes the three-way valve to move to the inlet position. In a preferred embodiment, the closing event causes the three-way valve to move to the outlet position.

While specific embodiments of the invention have been shown and described in detail to illustrate the application of the principles of the invention, it will be understood that the invention may be embodied otherwise without departing from such principles.

### LIST OF REFERENCE SIGNS

| | |
|---|---|
| 1 | Sample inlet, surrounds tube 31, belongs to input fluid guide unit |
| 2 | Linearly movable sealing cone, acting as a valve body and as a closure part, |
| | movable relative to the valve body seat 13, arranged upstream (first |
| | embodiment) or downstream (second embodiment) of the valve body seat 13 |
| 3 | Measuring chamber, receives a sample flowing in through the sample inlet 1, |
| | surrounds the sensor 12, is surrounded by the wall 40 |
| 4 | Rod, connects the sealing cone 2 with the solenoid 7, guided through the |
| | connecting sleeve 11 and guided through the measuring chamber 3, belongs to |
| | the mechanical connecting element |
| 5 | Bellows capable of generating a negative pressure and a positive pressure in the |
| | measuring chamber 3, is pulled apart and compressed by the plate 6, acts as a |
| | suction chamber |
| 6 | Plate, is able to pull apart and compress the bellows 5, acts as a chamber |
| | modifying element |
| 7 | Solenoid, linearly moves rod 4 parallel to its longitudinal axis, longitudinal |
| | axis, acts as actuator |
| 8 | Suction fluid connection between the measuring chamber 3 and the bellows 5 |
| 9 | Frame (rack) on which the sample inlet 1, the wall 40, the sensor 12 and the |
| | solenoid 7 are mounted |
| 10 | Outflow-side connection piece, attached to measuring chamber 3 |
| 11 | Connecting sleeve, through which the rod 4 is passed, firmly connected to the |
| | rod 4 and to the plate 6, belongs to the mechanical connecting element |
| 12 | Electrochemical sensor in the measuring chamber 3, comprises electrodes 20 |
| | and 21 and electrical contacts 33, 34, is capable of determining an indicator of |
| | the concentration of breath alcohol in the measuring chamber sample |
| 13 | Sealing ring around the rod 4, acts as a valve body seat and thus as a closure |
| | part seat for the sealing cone (valve body) 2, arranged downstream (first |
| | embodiment) or upstream (second embodiment) from the sealing cone 2 |
| 14 | Further sealing ring, arranged around the tube 16 |
| 15 | Cavity in connecting piece 16 |
| 16 | Connecting piece between the sample inlet 1 and the sealing cone 2, surrounds |
| | the cavity 15, comprises the parts 16.1 and 16.2, belongs to the input fluid |
| | guide unit |
| 16.1 | Smaller part of the connector 16 |
| 16.2 | Larger part of the connector 16 |
| 17 | Cover plate for the sensor 12 |
| 18 | Recess belonging to an input fluid connection between the cavity 15 and the |
| | measuring chamber 3 |
| 19 | Guide unit that guides the rod 4 linearly |
| 20 | Measuring electrode of the sensor 12, is contacted by the electrical contact 34 |
| 21 | Counter electrode of the sensor 12, is contacted by the electrical contact 33 |
| 25 | Stop element on the sample inlet 1, limits a movement of the mouthpiece 30 |
| | towards the measuring chamber 3. |
| 26 | Connecting element between the plate 6 and the solenoid 7 |
| 27 | Bolt, passing through a recess in the connecting element 26 and in a rod 4 of the |
| | solenoid element 7 |
| 28 | Electrolyte between the two electrodes 20 and 21 |
| 30 | Funnel-shaped mouthpiece, directs a breath sample A into the sample inlet 1 |
| 31 | Tube inside the sample inlet 1 |
| 32 | Inlet-side connection piece, attached to the measuring chamber 3, surrounds the |
| | larger part 16.2 |
| 33 | Electrical contacting of the counter electrode 21 |
| 34 | Electrical contacting of the measuring electrode 20 |
| 40 | Wall of measuring chamber 3 |
| 50 | Sensor arrangement, comprises a sensor 12 and a measuring chamber 3 |
| 60 | Control unit |
| 100 | Analyzer, includes mouthpiece 30, frame 9, sample inlet 1, measuring chamber |
| | 3, sensor 12, rod 4, valve 2, 13, actuator with solenoid 7 and connecting sleeve |
| | 11 |
| 103 | Measuring chamber, receives a sample flowing in through the sample inlet side |
| | opening Ö.e, surrounds the sensor 112, is surrounded by the wall 140 |
| 112 | Electrochemical sensor in the measuring chamber 103, comprises electrodes |
| | 120 and 121 and electrical contacts 133, 134, is capable of determining an |
| | indicator of the concentration of breath alcohol in the measuring chamber |
| | sample Pr |
| 120 | Measuring electrode of the sensor 112, is contacted by the electrical contact 134 |
| 121 | Counter electrode of the sensor 112, is contacted by the electrical contact 133 |
| 122 | Electrical connection between contacts 133 and 134 |
| 128 | Electrolyte between the two electrodes 120 and 121 |
| 129 | Electrical measuring resistance between the two electrodes 120, 121 |
| 133 | Electrical contacting of the counter electrode 121 |
| 134 | Electrical contacting of the measuring electrode 210 |
| 138 | Current sensor, measures the strength of the current flowing through the |
| | electrical connection 122 |
| 140 | Wall of measuring chamber 103 |
| 150 | Sensor arrangement, comprises a sensor 112 and a measuring chamber 103 |
| A | Breath sample to be analyzed for breath alcohol contains the measuring |
| | chamber sample, which is aspirated into the measuring chamber 3 |
| MA | Coinciding center axis of the measuring chamber 3 and the sensor 12 |
| MA1 | Coinciding center axis of the measuring chamber 103 and the sensor 112 |
| MP.1 | First measuring point, is in fluid connection with the cavity 15 or recess 18 and |
| | thereby in fluid connection with the mouthpiece 30 |
| MP.2 | Second measuring point, is in fluid connection with the measuring chamber 3 |
| Ö.a | Outlet-side opening in the housing, through which the measuring chamber |
| | sample Pr flows out of the measuring chamber 103 |
| Ö.e | Opening on the inlet side in the housing, through which the measuring |
| | chamber sample Pr flows into the measuring chamber 103 |
| Pr | Measuring chamber sample, which is that part of the breath sample emitted by |
| | the subject that enters the measuring chamber 103 |
| Sp | Circumferential gap between the sealing cone 2 and the inner wall of the cavity |
| | 15 |

What is claimed is:

1. An analyzer for analyzing a gas sample delivered by a subject for a predetermined substance, the analyzer comprises:
an input unit configured to input or receive the gas sample;
a measuring chamber, the analyzer being configured to at least temporarily provide an input fluid connection between the input unit and the measuring chamber;
a sensor configured to measure an indicator of an amount of the substance in a gas located in the measuring chamber and/or an indicator of a concentration of the substance in a gas located in the measuring chamber;
a suction chamber unit configured to be selectively transferred into a minimum volume state or a maximum volume state, the analyzer being configured to at least temporarily provide a suction fluid connection between the suction chamber unit and the measuring chamber;
a valve configured to be moved into a closing end position in which the valve interrupts the input fluid connection and to be moved into a releasing end position in which the valve releases the input fluid connection; and
a drive unit configured to selectively move the valve into the closing end position or into the releasing end position and to selectively transfer the suction chamber unit into the minimum volume state or into the maximum volume state, the drive unit being mechanically coupled to the valve and being mechanically coupled to the suction chamber unit such that:
a movement of the valve into the releasing end position is synchronized with a transfer of the suction chamber unit into the minimum volume state, and a movement of the valve into the closing end position is synchronized with a transfer of the suction chamber unit into the maximum volume state; or
a movement of the valve into the releasing end position is synchronized with a transfer of the suction chamber unit into the maximum volume state, and a movement of the valve into the closing end position is synchronized with a transfer of the suction chamber unit into the minimum volume state,
wherein the analyzer is configured such that a transfer of the suction chamber unit into the maximum volume state causes gas to be sucked out of the input unit through the input fluid connection into the measuring chamber.

2. An analyzer according to claim 1, wherein:
the drive unit comprises: an actuator; and a mechanical valve connecting element;
the valve comprises a closure part; and a closure part seat; and
the valve connecting element mechanically connects the actuator to the closure part.

3. An analyzer according to claim 1, wherein the measuring chamber is located between the input unit and the suction chamber unit.

4. An analyzer according to claim 1, wherein:
the drive unit comprises an actuator mechanically coupled to the valve and mechanically coupled to the suction chamber unit; and
the suction chamber unit is located between the measuring chamber and the actuator.

5. An analyzer according to claim 1, wherein:
the suction chamber unit comprises a suction chamber with variable volume and a chamber modifying element;
the drive unit comprises an actuator and a mechanical suction chamber connecting element;
the suction fluid connection connects the suction chamber to the measuring chamber;
a movement of the chamber modifying element relative to the suction chamber causes the volume of the suction chamber to be changed; and
the suction chamber connecting element mechanically connects the actuator to the chamber modifying element.

6. An analyzer according to claim 5, wherein
the drive unit comprises: an actuator; and a mechanical valve connecting element;
the valve comprises a closure part; and a closure part seat; and
the valve connecting element mechanically connects the actuator to the closure part and comprises the suction chamber connecting element.

7. An analyzer according to claim 1, further comprising a volume flow sensor configured to measure an indicator of a volume flow of a gas through the input fluid connection into the measuring chamber, wherein the analyzer is configured to actuate the drive unit to move the valve into the closing end position, the actuation is performed depending on the measured volume flow.

8. An analyzer according to claim 1, further comprising a fluid guide unit, wherein:
the valve comprises a closure part;
the fluid guide unit surrounds the closure part;
an intermediate space is present between the fluid guide unit and the closure part; and
the input fluid connection passes through the fluid guide unit and includes the intermediate space.

9. An analyzer according to claim 1, wherein the analyzer is configured such that the transfer of the suction chamber unit into the minimum volume state causes gas to be conveyed through the input fluid connection out of the measuring chamber.

10. An analyzer according to claim 1, further comprising an input fluid guide unit, wherein:
the input unit is configured to be connected to the input fluid guide unit;
the input fluid connection passes through the input fluid guide unit; and
the input fluid guide unit fully or at least partially surrounds the valve.

11. A process for analyzing a gas sample delivered by a subject for a predetermined substance, the process comprising the steps of:
providing an analyzer, wherein the analyzer comprises an input unit; a measuring chamber; a sensor; a suction chamber unit which can be selectively transferred into a minimum volume state or a maximum volume state; a valve and a drive unit, wherein the analyzer at least temporarily provides an input fluid connection between the input unit and the measuring chamber and at least temporarily provides a suction fluid connection between the suction chamber unit and the measuring chamber;
initially the valve being in a closing end position in which the valve interrupts the input fluid connection;
inputting the gas sample into the input unit or receiving the gas sample by the input unit;
with the drive unit, moving the valve into a releasing end position in which the valve releases the input fluid connection;
with the drive unit, transferring the suction chamber unit into the maximum volume state causing gas to be sucked out of the input unit through the input fluid connection into the measuring chamber;
subsequent to the step of transferring the suction chamber unit into the maximum volume state, with the drive unit, transferring the suction chamber unit into the minimum volume state causing gas to be conveyed from the suction chamber unit through the suction fluid connection into the measuring chamber and gas is thereby expelled from the measuring chamber;
with the drive unit, moving the valve back into the closing end position; and
with the sensor, measuring an indicator of a concentration of the substance in the gas located in the measuring chamber and/or an indicator of an amount of the substance in the gas located in the measuring chamber,
wherein either:
the step of moving the valve into the releasing end position and the step of transferring the suction chamber unit into the minimum volume state are performed simultaneously, and the step of moving back the valve into the closing end position and the step of transferring the suction chamber unit into the maximum volume state are performed simultaneously, or
the step of moving the valve into the closing end position and the step of transferring the suction chamber unit into the minimum volume state are performed simultaneously, and the step of moving back the valve to the releasing end position and the step of transferring the suction chamber unit to the maximum volume state are performed simultaneously.

12. A process according to claim 11, wherein:
an event is detected that the input of the gas sample into the input unit is started, wherein the step of moving the valve into the releasing end position is started;
if a predefined period of time has elapsed since the gas sample entered the input unit and/or if an opening event has occurred after the start of gas sample input, wherein the opening event depends on an indicator of the volume or amount of the gas sample previously input into the input unit.

13. A process according to claim 11, wherein at least once an indicator of the amount of gas that has so far flowed into the measuring chamber after the start of the step of moving the valve into the releasing end position is measured, and upon the measured amount having reached a predetermined quantity limit, the step of moving the valve back into the closing end position is triggered.

14. A process according to claim 11, wherein before carrying out the process, the suction chamber unit is in the maximum volume state, and the step of transferring the suction chamber unit to the minimum volume state is carried out before the step of transferring the suction chamber unit to the maximum volume state.

15. A process according to claim 11, wherein before carrying out the process, the suction chamber unit is in the minimum volume state, and the step of transferring the suction chamber unit into the maximum volume state is carried out before the step of transferring the suction chamber unit into the minimum volume state.

16. A process according to claim 11, wherein by means of a mechanical coupling of the drive unit with the valve and with the suction chamber unit, the drive unit moves the valve into the one end position and the drive unit transfers the suction chamber unit into the minimum or into the maximum volume state.
